(19) United States
(12) Patent Application Publication
(10) Pub. No.: US 2026/0306727 A1
(43) Pub. Date: Oct. 8, 2026

(54) PUMPING SYSTEM AND METHOD FOR HOME HEALTHCARE DEVICES

(71) Applicant: Michael Anthony Dorantes, Santa Monica, CA (US)

(72) Inventor: Michael Anthony Dorantes, Santa Monica, CA (US)

(21) Appl. No.: 19/171,170

(22) Filed: Apr. 4, 2025

(51) Int. Cl. A61M 1/26; A61M 1/14; A61M 16/16; F04B 43/12

(52) U.S. Cl. CPC A61M 1/262; A61M 1/155; A61M 1/267; A61M 16/162; F04B 43/12; A61M 2205/103

(57) ABSTRACT

A system for draining and refilling home healthcare equipment. The system may include a suction tube portion insertable into a container of liquid. The system may include a discharge tube portion insertable into a liquid receptacle or drain may include a peristaltic pump connected to the suction tube portion and to the discharge tube portion, the peristaltic pump operable to pump liquid from the container of liquid to the liquid receptacle or drain, the peristaltic pump being operable by a user while stationary.

### BACKGROUND

### Field

The present disclosure is directed to a pumping system and method for home healthcare devices, such as continuous positive airway pressure (CPAP) machines and dialysis bags.

### Description of the Related Art

Home health care devices and in-home medical treatments are widely used and administered throughout the country. These devices are typically operated by the user or by a home health care professional. Such devices include Continuous Positive Airway Pressure (CPAP) machines which can be equipped with humidifiers that provide humidified air to users, preventing nasal and throat dryness during extended use. These CPAP humidifiers require a water source which is provided by way of a distilled water tank, often located within the CPAP system. These tanks need to be refilled frequently with repeated use.

Additionally, peritoneal dialysis treatment can be administered at home. Home-based dialysis treatment involves collecting waste dialysate in seal bags which require frequent disposal or draining.

### SUMMARY

Accordingly, there is an unmet need for a system and method that makes the refilling of CPAP humidifiers and the draining of dialysis bags more manageable for elderly or infirm users.

In some aspects, the techniques described herein relate to a method for pumping liquid into a home healthcare device, including: filling a container with water; inserting a suction tube portion into the container; inserting a discharge tube portion into a humidifier reservoir; and pumping with a pump water from the container to the humidifier reservoir with water to refill the humidifier reservoir, while a user operates the pump from a stationary bed-ridden position.

### BRIEF DESCRIPTION OF THE DRAWINGS

FIG. 1 shows an embodiment of a CPAP humidifier refilling system

FIG. 2 shows an embodiment of distilled water reservoir and pump configuration for a CPAP humidifier refilling system.

FIG. 3 shows an embodiment of a dialysis bag draining system.

FIG. 4 shows an embodiment of a locking mechanism.

FIG. 5 shows an embodiment of an actuator tube used in conjunction with the dialysis bag draining system.

FIG. 6 shows a method for implementing a CPAP humidifier refilling system.

FIG. 7 shows a method for implementing a dialysis bag draining system.

### DETAILED DESCRIPTION

Disclosed herein are embodiments of pumping systems and methods for home healthcare devices (such as CPAP machines and dialysis bags), which can include CPAP humidifier refilling systems and dialysis bag draining systems.

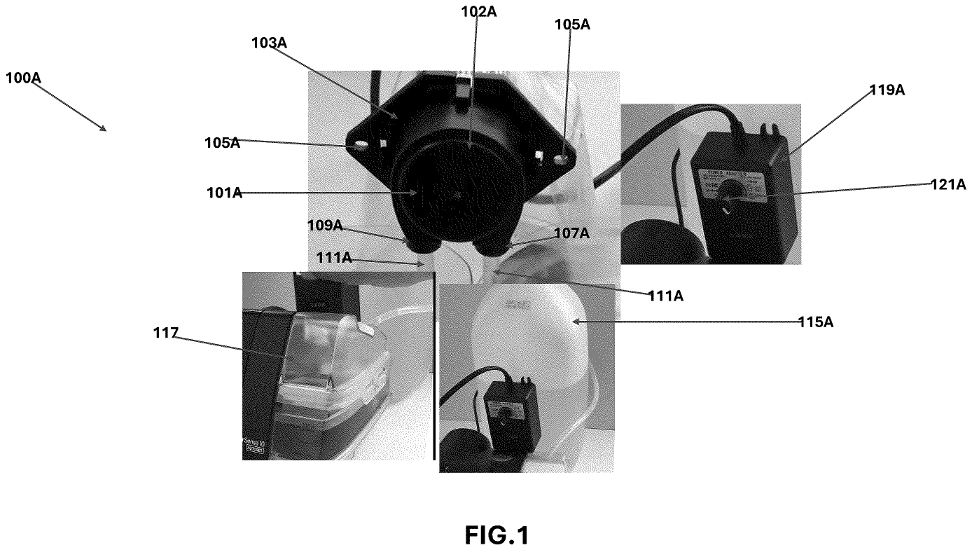

FIG. 1 shows an embodiment of a pumping system for a CPAP machine, specifically a CPAP humidifier refilling system 100A where a pump 101A (e.g., peristaltic pump) is used to pump distilled water from a water container 115A into a humidifier reservoir 117 of a CPAP machine. The pump 101A may be enclosed in a housing 102A. The housing may form a flange 103A which may have holes 105A bored through and about any portion of its surface (which can facilitate mounting of the pump 101A to a surface). The pump 101A may be a peristaltic pump and have a rotating roller which squeezes a tube 111A creating a vacuum that pulls liquid into the tube 111A. Tube 111A may be up to twelve feet in length or longer. The pump 101A may have a suction port 107A and a discharge port 109A. The tube 111A may extend from the water container 115A into the suction port 107A. Then the tube 111A may extend out of the discharge port 109A and into the humidifier reservoir 117. In one example, a single tube 111A extends from the water container 115A, through the suction port 107A, through the pump 101A, through the discharge port 109A and to the humidifier reservoir 117. In another example, the CPAP humidifier refilling system 100A uses two separate tubes, one tube extending from the water container 115A to the suction port 107A and a separate tube extending from the discharge port 109A to the humidifier reservoir 117, the pump 101A having a separate tube portion or conduit therein that connects to the two tubes via the suction port 107A and the discharge port 109A. The pump 101A may receive power from a power controller 119A, which can connect (via a power cord or electric cord) to a wall outlet. The power controller 119A may have a user interface module 121A that is manually actuatable and allows the user to control the flow rate of the CPAP humidifier refilling system 100A. In some embodiments, the system 100A may be battery operated. In the illustrated example, the pump 101A is a peristaltic pump. In other examples, the pump 101A can be a gear pump, a diaphragm pump or other suitable pump types. In an example where the CPAP humidifier system uses a diaphragm pump, then the system may use two separate tubes, one tube extending from the water container 115A to the suction port 107A and a separate tube extending from the discharge port 109A to the humidifier reservoir 117. In some embodiments the pump 101A may have dimensions which may include as being 5-15 centimeters in length, 4-12 centimeters in width, and 3-10 centimeters in height. The CPAP humidifier refilling system 100A may be placed on and operated from a tabletop, or any other suitable surface, which is at some distance away from a home health care device and/or the user. Alternatively, the system 100A may be placed on and operated from a tabletop, or any other suitable surface, which is bedside or in close proximity to the home health care device and/or user.

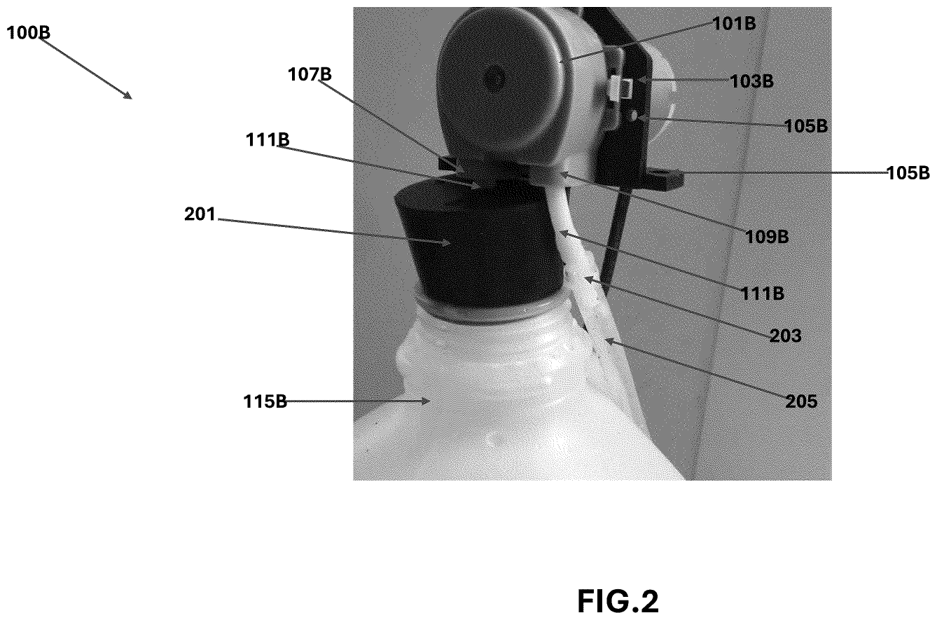

FIG. 2 shows an embodiment of a CPAP humidifier refilling system 100B which may be a variation of CPAP humidifier refilling system 100A. CPAP humidifier refilling system 100B includes a pump 101B that, in one example may be a peristaltic pump and have a rotating roller that squeezes a tube 111B creating a vacuum that pulls distilled water from a container 115B and into a humidifier reservoir 117. The pump 101B may be enclosed in a housing 102B. The housing may form a flange 103B which may have holes 105B bored through and about any portion of its surface. The pump 101B may have a suction port 107B and a discharge port 109B. The tube 111B may have a tube connecter 203 which can be used to connect a tube extension 205 to the tube 111B. The tube connector 203 and tube extension 205 can be included on the suction side, the discharge side, or both the suction side and discharge side of the tube 111B. In other examples, the pump 101B can be a gear pump, a diaphragm pump or other suitable pump types. In an example where the CPAP humidifier system uses a diaphragm pump, then the system may use two separate tubes, one tube extending from the water container 115B to the suction port 107B and a separate tube extending from the discharge port 109B to the humidifier reservoir 117. The system may include a stopper 201 that can be coupled to (e.g., at least partially inserted into) the water container 115B to ensure a hermetically sealed connection between the water container 115B and the tube 111B. In some embodiments the pump 101B may be mounted on top of the stopper 201 as shown in FIG. 2.

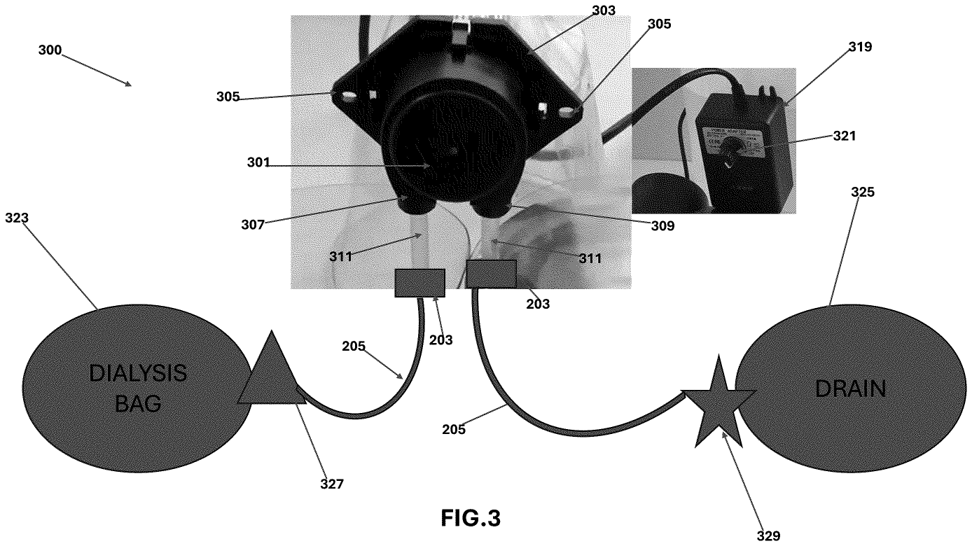

FIG. 3 shows a pumping system for dialysis, specifically a dialysis bag draining system 300 where a pump 301 (e.g., peristaltic pump) is used to pump waste fluid from a dialysis bag 323 into a drain 325 to dispose of the waste fluid. The pump 301 may be enclosed in a housing 302. The housing may form a flange 303 which may have holes 305 bored through and about any portion of its surface. The pump 301 may be a peristaltic pump and have a rotating roller which squeezes a pump tube 311 creating a vacuum that pulls liquid into the pump tube 311. The pump 301 may have a suction port 307 and a discharge port 309. The pump tube 311 may extend from the dialysis bag 323 into the suction port 307. Then the pump tube 311 may extend out of the discharge port 309 and into the drain 325. In one example, a single tube 311 extends from the dialysis bag 323, through the suction port 307, through the pump 301, through the discharge port 309 and to the drain 325. In another example, the dialysis bag draining system 300 uses tube extensions 205. As shown in FIG. 3, a tube extension 205 can be attached to the dialysis bag 323 via a locking mechanism 327. The tube extension 205 can then extend from the locking mechanism 327 to a tube connector 203 that connects the tube extension 205 to the pump tube 311. As shown in FIG. 3, the pump tube 311 extends through the suction port 307, through the pump 301, and out of the discharge port 309. The pump tube 311 may be connected to a tube extension 205 via a tube connector 203. The tube extension 205 may then extend and connect to the drain 325 via a mounting mechanism 329.

The mounting mechanism 329 can include but is not limited to a hook, brace, or clamp. The tube extensions 205 can vary in length and be the same or different lengths relative to each other.

In some embodiments of system 300, the pump 301 may be mounted, by way of the holes 305, to a surface or structure that is in close proximity to the home healthcare device and/or the user. In other embodiments, the pump 301 may be suspended between the dialysis bag 323 and a drain 325 in any manner which is suitable for operation. Advantageously, this will add to the adaptability of installing and operating the system in different home healthcare or hospital settings. The pump 301 may receive power from a power controller 319, which can connect (via a power cord or electric cord) to a wall outlet. The power controller 319 may have a user interface module 321 that is manually actuatable and allows the user to control the flow rate of the of dialysis bag draining system 300. In other examples, the system 300 may be battery operated. In the illustrated example, the pump 301 is a peristaltic pump. In other examples, the pump 301 can be a gear pump, a diaphragm pump or other suitable pump types. In an example where the pumping system 300 uses a diaphragm pump then those skilled in the art would understand that the system would use two separate tubes, one tube extending from the dialysis bag 323 to the suction port 307 and a separate tube extending from the discharge port 309 to the drain 325. In the illustrated example, the drain 325 may be a household sink, bathtub, toilet, or any other suitable liquid drain.

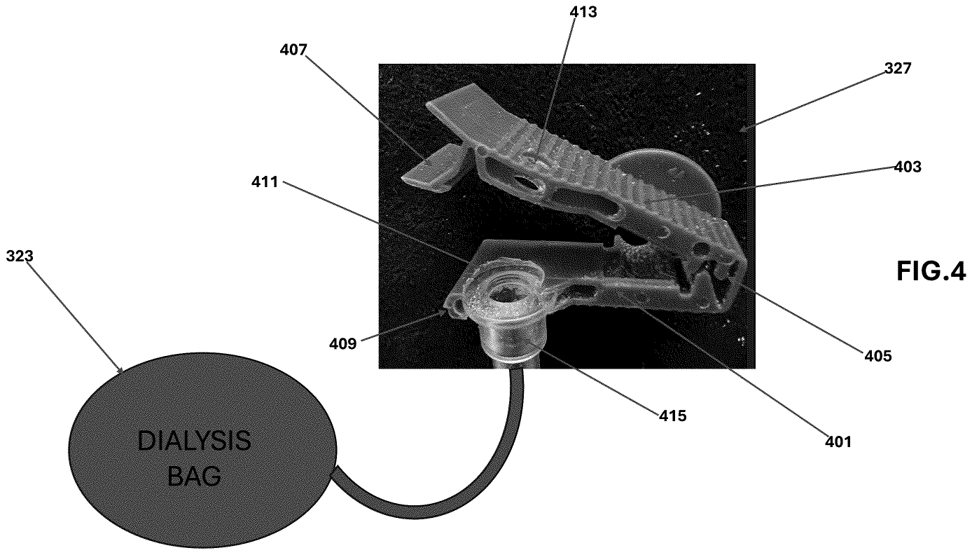

FIG. 4 depicts an example of a locking mechanism 327 shown in FIG. 3. The locking mechanism can be used to attach the pump tube 311 or the tube extension 205 to the dialysis bag. The locking mechanism 327 may have a bottom arm 401, a top arm 403, a hinge 405, a latch 407, a lip 409, an inlet port 411, and outlet port 413. In one embodiment, the locking mechanism 327 may be connected to the dialysis bag via a drain port tube 415. The drain port tube may be inserted into the inlet port 411. The top arm 403 can pivot towards the bottom arm 401 via the hinge 405. The hinge 405 may be integrated into the locking mechanism 327 forming one continuous structure or it may be a separate component. The latch 407 may be formed to fit around the lip 409, which facilitates the clamping function of the locking mechanism 327. The pump tube 311 or the tube extension 205 may then connect to the locking mechanism 327 by way of the outlet port 413.

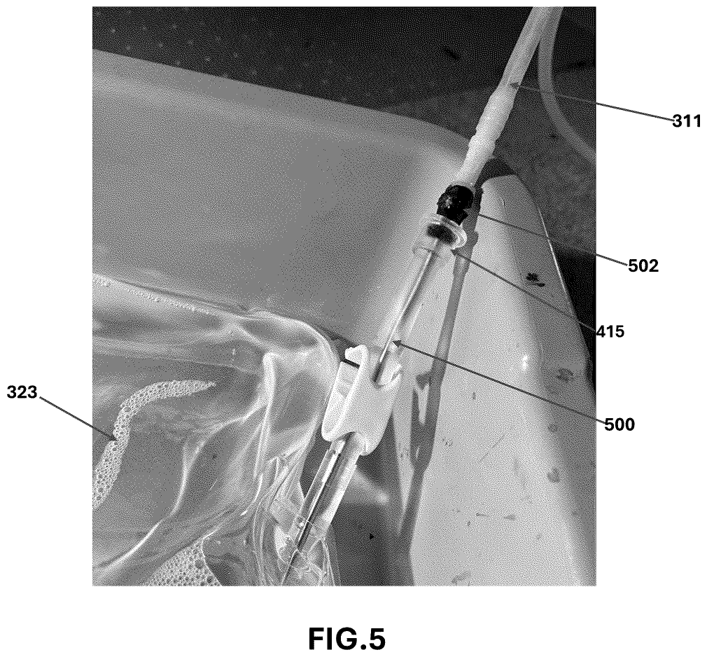

FIG. 5 depicts another embodiment of the suction side of the dialysis bag draining system of FIG. 3. The figure includes the pump tube 311, a compression fitting 502, the drain port tube 415, the dialysis bag 323 and an actuator tube 500. The actuator tube 500 may be hermetically coupled to the suction portion of the pump tube 311 via the compression fitting 502. The suction portion of the pump tube 311 may then be coupled to the drain port tube 415 via the compression fitting 502. Advantageously, this configuration allows for sufficient drainage without the use of a locking mechanism 327.

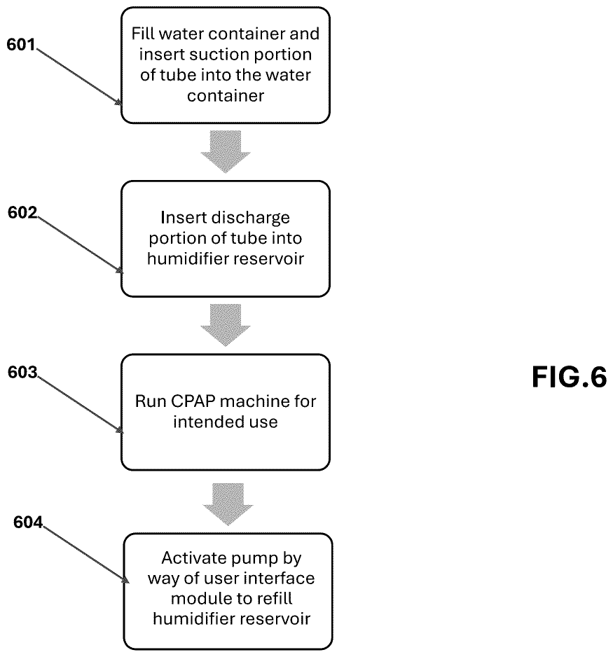

FIG. 6 describes a method for using a pumping system for a CPAP machine, specifically the CPAP humidifier refilling system 100A and or 100B. At step 601, a user will fill the water container 115 with liquid (e.g., with distilled water) to a desired amount. The container 115 may be placed in close proximity to the other components of the system or wherever is convenient for the user (e.g., at a bedside table). Then, a user will insert the suction portion of the tube 111 into the water container 115. The suction portion of the tube may be inserted first through the stopper 201 and then into the water container 115. Beneficially, the stopper 201 may provide a seal between the tube and the water container which may improve pumping efficacy of the system and prevent spillage of any associated liquid from the container 115. At step 601, a tube extension 205 may instead be inserted into the water container 115 and then the tube extension may be connected to the suction portion of the tube 111 by way of the tube connecter 203. Beneficially, the tube connecter 203 and tube extension 205 configuration allows users to transport water from a water container that may be some distance away from the rest of the system. This would reduce the need for carrying the water reservoir from the initial water source to a location that is near the other components of the system, which may be difficult for elderly or infirm users. At step 602, the user may insert the discharge portion of the tube 111 in a humidifier reservoir 117 of a CPAP machine. A tube extension 205 may instead be inserted into the humidifier reservoir 117 and then the tube extension 205 may be connected to the discharge portion of the tube 111 by way of the tube connecter 203. At step 603, the user would sleep while the CPAP machine is in use. As the humidifier reservoir depletes its water content, the user, at step 604, can activate the system by way of the user interface module 121 of the power controller 119. The user interface module can consist of a dial, slider, lever or any suitable selector that allows the user to select a desired flow rate for the system. Advantageously, the CPAP humidifier refilling system 100 allows a user to fill the humidifier reservoir 117 without having to get out of bed, which may be difficult for elderly or infirm users. Additionally, the CPAP humidifier refilling system 100 allows the user to fill the humidifier reservoir 117 on their own, without requiring assistance from another person (e.g., a caregiver, nurse, etc.).

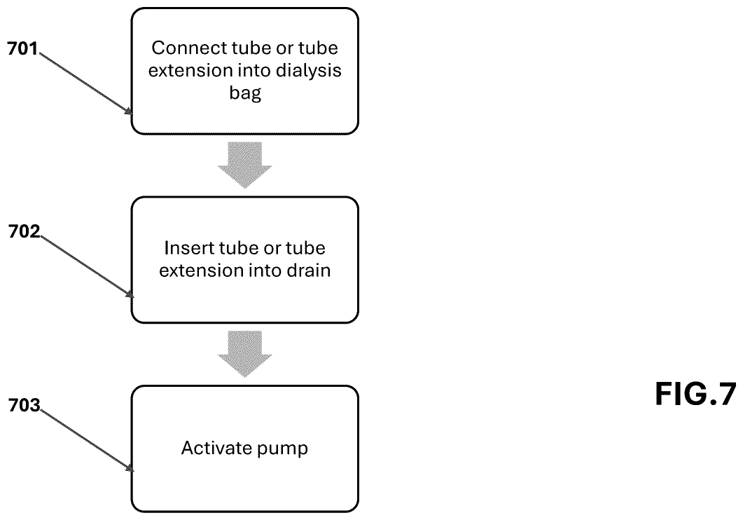

FIG. 7 describes a method for using a pumping system for dialysis, specifically a dialysis bag draining system 300. At step 701 a user will connect the suction portion of the pump tube 311 directly to a dialysis bag 323. Alternatively, the suction portion of the pump tube 311 can first be connected to the tube extension 205 by way of the tube connecter 203 and then connected to the dialysis bag 323. The pump tube 311, or the tube extension 205, can be connected to the dialysis bag 323 by a locking mechanism 327. The pump tube 311 or the tube extension 205 can be up to twelve feet in length or longer. At step 702, a user will connect the discharge portion of the pump tube 311 directly to a drain 325. Alternatively, the discharge portion of the pump tube 311 can first be connected to the tube extension 205 by way of the tube connecter 203 and then connected to the drain 325. The drain may include a household sink, bathtub, toilet, or any suitable liquid drain. The pump tube 311, or the tube extension 205, can be connected to the drain 325 by a mounting mechanism 329. The mounting mechanism 329 can include but is not limited to a hook, brace, or clamp. At step 703, a user will activate the pump by engaging the user interface module 321 which can control the speed at which the pump removes waste from the dialysis bag 323 and into the drain 325. Users undergoing dialysis are typically elderly or physically debilitated; thus, the method described above advantageously would obviate the cumbersome task of physically carrying a dialysis bag (which may be heavy) to a drain and emptying the dialysis bag into a drain after repeated treatment sessions.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of the stated amount. As another example, in certain embodiments, the terms “generally parallel” and “substantially parallel” refer to a value, amount, or characteristic that departs from exactly parallel by less than or equal to 15 degrees.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Of course, the foregoing description is that of certain features, aspects and advantages of the present invention, to which various changes and modifications can be made without departing from the spirit and scope of the present invention. Moreover, the devices described herein need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or sub combinations of these specific features and aspects of embodiments may be made and still fall within the scope of the invention. Accordingly, it should be understood that various features and aspects of the disclosed embodiments can be combined with or substituted for one another in order to form varying modes of the discussed devices.

What is claimed is:

1. A method for pumping liquid in a home healthcare device, comprising:
filling a container with water;
inserting a suction tube portion into the container;
inserting a discharge tube portion into a humidifier reservoir; and
pumping with a pump water from the container to the humidifier reservoir with water to refill the humidifier reservoir, a user operating the pump while stationary.

2. The method of claim 1, wherein the pump is a peristaltic pump.

3. The method of claim 2, wherein the pump is 5-15 centimeters in length, 4-12 centimeters in width, and 3-10 centimeters in height.

4. The method of claim 2, wherein the suction tube portion and the discharge tube portion are part of a same tube.

5. The method of claim 1, wherein the pump is mounted onto the container by way of a stopper.

6. The method of claim 1, wherein the pump is connected to the suction tube via a first tube connecter and the pump is connected to the discharge tube via a second tube connecter.

7. The method of claim 1, wherein each of the suction tube and the discharge tube is at least 15 centimeters.

8. The method of claim 1, wherein a flowrate of the pump is controlled with a user interface module of a power controller wherein the power controller is connected to the pump.

9. A method for pumping liquid in a home healthcare device, comprising:
inserting a suction tube portion into a dialysis bag;
extending a discharge tube portion to a drain; and
pumping with a pump liquid from the dialysis bag to the drain, a user operating the pump while stationary.

10. The method of claim 9, wherein the pump is a peristaltic pump.

11. The method of claim 10, wherein the pump is 5-15 centimeters in length, 4-12 centimeters in width, and 3-10 centimeters in height.

12. The method of claim 10, wherein the suction tube portion and the discharge tube portion are part of a same tube.

13. The method of claim 9, wherein inserting the suction tube into a dialysis bag comprises attaching the suction to the dialysis bag via a clamp.

14. A pumping system for pumping liquid in a home healthcare device, comprising:
a suction tube portion insertable into a container of liquid;
a discharge tube portion insertable into a liquid receptacle or drain; and
a peristaltic pump connected to the suction tube portion and to the discharge tube portion, the peristaltic pump operable to pump liquid from the container of liquid to the liquid receptacle or drain, the peristaltic pump being operable by a user while stationary.

15. The system of claim 14, wherein the suction tube portion and the discharge tube portion are part of a same tube.

16. The system of claim 14, wherein the container of liquid is a container of distilled water.

17. The system of claim 14, wherein the container of liquid is a dialysis bag.

18. The system of claim 14, wherein the liquid receptacle is a humidifier tank of a CPAP machine.

19. The system of claim 14, wherein the pump is configured to be mountable or suspended in between the container of liquid and the liquid receptacle or drain.

20. The system of claim 14, wherein a flowrate of the pump is controlled with a user interface module of a power controller wherein the power controller is connected to the pump.
